(19) United States
(12) Patent Application Publication
(10) Pub. No.: US 2012/0196280 A1
(43) Pub. Date: Aug. 2, 2012

(54) MICROFABRICATED DEVICE FOR METERING AN ANALYTE

(75) Inventors: Frank Karlsen, Klokkarstua (NO); Anja Gulliksen, Klokkarstua (NO); Lars Anders Solli, Klokkarstua (NO)

(73) Assignee: NORCHIP A/S, Klokkarstua (NO)

(21) Appl. No.: 13/384,379

(22) PCT Filed: Jul. 16, 2010
(86) PCT No.: PCT/EP2010/004371

§ 371 Date: Apr. 16, 2012

(30) Foreign Application Priority Data
Jul. 17, 2009 (GB) 0912509.7

(51) Int. Cl. C12Q 1/68; C12M 1/40

(52) U.S. Cl. 435/6.1; 435/289.1

(57) ABSTRACT

The present invention relates to a microfabricated device for metering an analyte comprising a nucleic acid sequence into a plurality of parallel reaction chambers for nucleic acid sequence amplification. The present invention further provides a method of metering an analyte into a plurality of parallel reaction units of an integrated microfabricated device.

The present invention relates to a microfabricated device for metering an analyte comprising a nucleic acid sequence into a plurality of parallel reaction chambers for nucleic acid sequence amplification. The present invention further provides a method of metering an analyte into a plurality of parallel reaction units of an integrated microfabricated device.

### BACKGROUND TO THE INVENTION

Nucleic acid amplification is a powerful analytical tool. The first amplification technique that was developed was the Polymerase Chain Reaction (PCR) and this technique is still the most widely used amplification technique. However, other techniques have been developed to overcome particular drawbacks of PCR. Examples of other techniques include self-sustained sequence replication (3SR), strand-displacement amplification (SDA), the ligase chain reaction (LCR), QB replicase amplification (QBR), ligation activated transcription (LAT), nucleic acid sequence-based amplification (NASBA) and the repair chain reaction (RCR).

Nucleic acid amplification has found numerous practical applications. For example, it can be used to analyze DNA and/or RNA isolated and purified from bacterial cells and virus particles. Thus, nucleic acid amplification has been used in many areas of technology such as, for example, diagnostics, environmental monitoring, forensics and molecular biology research.

Whatever amplification technique is used, nucleic acid amplification is usually carried out in a laboratory setting by mixing enzymes, primers and an analyte containing nucleic acids together and heating the mixture as necessary to amplify the nucleic acids. The amplification may be carried out in, for example, wells of a microtiter plate.

The selection of the primers used in the amplification reaction usually determines which nucleic acids are amplified. Therefore, a convenient method of carrying out multiple analyses on a single sample is to pipette aliquots of a single sample into different wells of a microtiter plate where each well contains a different primer. The acts of loading samples and reagents onto a microtiter plate may be performed manually and performed by a trained laboratory technician or may be automated and be carried out by a specially designed robot.

Recently, interest has grown in the possibility of providing microfabricated (microfluidic) systems to carry out amplification reactions. One advantage of using microfabricated systems is that amplification is possible on a much smaller sample volume than other techniques. However, there are practical drawbacks associated with the use of a smaller volume sample resulting from the difficulty in handling a small sample volume.

In microfabricated reaction chamber systems, it is known to carry out multiple nucleic acid amplification reactions on a single sample by loading a sample into multiple sets of reaction chambers. One approach has been to adapt the macro-scale approach to loading microtiter wells with pipettes to the micro-scale. For example, U.S. Pat. No. 6,521,181 describes the use of a microinjector to inject a sample into an array of 384 individual-controlled PCR reaction chambers.

A different approach, for example that taken in WO 02/22265, has been to arrange reaction chambers in parallel connected to a common inlet port. This approach is illustrated in FIG. 1. In use, WO 02/22265 suggests one method of loading its sample is by use of a pump (4) connected to the inlet port. Thus, the pump ‘pushes’ its sample so that it is loaded into the parallel arrangement of reaction chambers (1, 2 and 3). This approach is developed in WO 03/060157, which suggests the loading of a sample into its system using one or more variable volume chambers.

Of the known nucleic acid amplification techniques, NASBA is an example of an amplification technique that can be used to produce RNA amplification products (in contrast, PCR is generally used to produce DNA amplification products). It is capable of yielding an RNA amplification of a billion fold in 90 minutes. It is suited to the amplification and detection of, for example, genomic, ribosomal or messenger RNA. One advantage of amplifying RNA analytes rather than DNA analytes is that the technique's application range is extended from the identification of biological targets as required in, for example, viral diagnostics to the indication of actual biological activity, such as gene expression and cell viability.

NASBA's adaptation to RNA amplification is accompanied by other differences between NASBA and PCR. For example, PCR typically requires the thermal cycling of its analyte in order to de-hybridize its DNA products from their complimentary strands before further amplification is possible. In contrast, NASBA typically does not require this thermal cycling because its RNA products are single stranded and the amplification step in NASBA may be carried out at a single temperature, i.e. isothermally (typically at about 41° C.). This isothermal temperature is typically lower than the temperatures usually required for PCR, and this is reflected by the choice of enzymes for NASBA, which are typically denatured by the temperatures used in PCR for thermal cycling.

Turning to the specifics of NASBA, NASBA technology is discussed, for example, in Nature volume 350 pages 91 and 92. Briefly, nucleic acid amplification in NASBA is accomplished by the concerted enzyme activities of AMV reverse transcriptase, RNase H, and T7 RNA polymerase, together with a primer pair, resulting in the accumulation of mainly single-stranded RNA that can readily be used for detection by hybridization methods. The application of an internal RNA standard to NASBA results in a quantitative nucleic acid detection method with a dynamic range of four logs but which needed six amplification reactions per quantification. This method is improved dramatically by the application of multiple, distinguishable, internal RNA standards added in different amounts and by electrochemiluminesence (ECL) detection technology.

This one-tube quantitative (Q) NASBA needs only one step of the amplification process per quantification and enables the addition of the internal standards to a clinical sample in a lysis buffer prior to the actual isolation of the nucleic acid. This approach has the advantage that the nucleic acid isolation efficiency has no influence on the outcome of the quantitation, which in contrast to methods in which the internal standards are mixed with a wild-type nucleic acid after its isolation from the clinical sample. Quantitative NASBA is discussed in Nucleic Acid Research (1998) volume 26, pages 2150-2155.

Post-NASBA product detection, however, can still be a labour-intensive procedure, normally involving enzymatic bead-based detection and electrochemiluminescent (ECL) detection or fluorescent correlation spectrophotometry. However, as these methodologies are heterogeneous or they require some handling of sample or robotic devices that are currently not cost-effective they are relatively little used for high-throughput applications. A homogeneous procedure in which product detection is concurrent with target amplification by the generation of a target-specific signal would facilitate large-scale screening and full automation. Recently, a novel nucleic acid detection technology, based on probes (molecular beacons) that fluoresce only upon hybridization with their target, has been introduced.

Molecular beacons are single-stranded oligonuclotides having a stem-loop structure. The loop portion contains a sequence complementary to the target nucleic acid, whereas the stem is unrelated to the target and has a double-stranded structure. One arm of the stem is labelled with a fluorescent dye, and the other arm is labelled with a non-fluorescent quencher. In an isolated state the probe does not produce fluorescence because absorbed energy is transferred to the quencher and released as heat.

When the molecular beacon hybridizes to its target it undergoes a conformational change that separates the fluorophore and the quencher, and the bound probe fluoresces brightly. Molecular beacon probes are discussed, for example, in U.S. Pat No. 6,037,130 and in Nucleic Acids Research, 1998, vol. 26, no. 9.

Even the one tube quantitative Q-NASBA process generally requires at least two steps, typically a first primer annealing step carried out at about 65 degrees Celsius followed by an amplification and detection step carried out at about 41 degrees Celsius. The enzymes required for the second step would be denatured by the elevated temperature required for the first step, so must be added once the temperature of the process components has fallen sufficiently. Furthermore, as for most nucleic acid sequence amplification and detection processes, NASBA requires reagents specific to the target nucleic acid sequence to be used. To carry out simultaneous analysis of a DNA/RNA sample for a number of different target nucleic acid sequences generally requires the handling of a large number of different reagent sets, each requiring separate handling and use in separate test tubes.

### SUMMARY OF THE INVENTION

The present invention provides a method of metering an analyte in a microfabricated device into a plurality of reaction units arranged in parallel and connected to a common inlet port, the method comprising:

providing a microfabricated device comprising: (a) a common inlet port, (b) a supply channel connected to the common inlet port, and (c) a plurality of reaction units connected in parallel to the common inlet port, each reaction unit comprising: (c1) a metering channel having a first end connected to the supply channel and a second end, (c2) a first reaction chamber, and (c3) a first valve directly connected to the second end of the metering channel and separating the metering channel from the first reaction chamber;

loading an analyte into the common inlet port,

allowing the analyte to enter the supply channel and then into each of the metering channels up to each of the first valves,

causing or allowing any analyte remaining in the supply channel to be drawn down the supply channel past and away from the first ends of each of the metering channels; and then

causing or allowing the analyte metered in each metering channel to pass through each valve into each of the first reaction chambers.

The present invention further provides a microfabricated device for carrying out nucleic acid sequence amplification on an analyte, the device comprising:

(a) a common inlet port,

(b) a supply channel connected to the common inlet port, and

(c) a plurality of reaction units connected in parallel to the common inlet port, each reaction unit comprising: (c1) a metering channel having a first end connected to the supply channel and a second end, (c2) a first reaction chamber, and (c3) a first valve connected to the second end of the metering channel and separating the metering channel from the first reaction chamber.

### DESCRIPTION OF THE FIGURES

The invention will be described with reference to the following figures, which are provided by way of example:

FIG. 1 is an illustration of the approach taken in WO 02/22265 to provide a sample to a parallel set of reaction chambers.

FIG. 2 shows a device according to the present invention.

FIG. 3 illustrates the measurement of an angle of deviation of a dimension of a channel from the direction of the flow path, the direction of the flow path being indicated by the arrow.

FIG. 4 shows one structure adapted for spotting a hydrophobic material into a constriction in a flow channel.

FIG. 5 shows a detailed device according to the present invention.

FIG. 6 shows a detail of storage reagent chambers and a sample loading chamber/mixing unit.

FIG. 7 shows typical dimensions of a device according to the invention.

FIGS. 8 and 9, 10 and 11 show devices manufactured according to the invention.

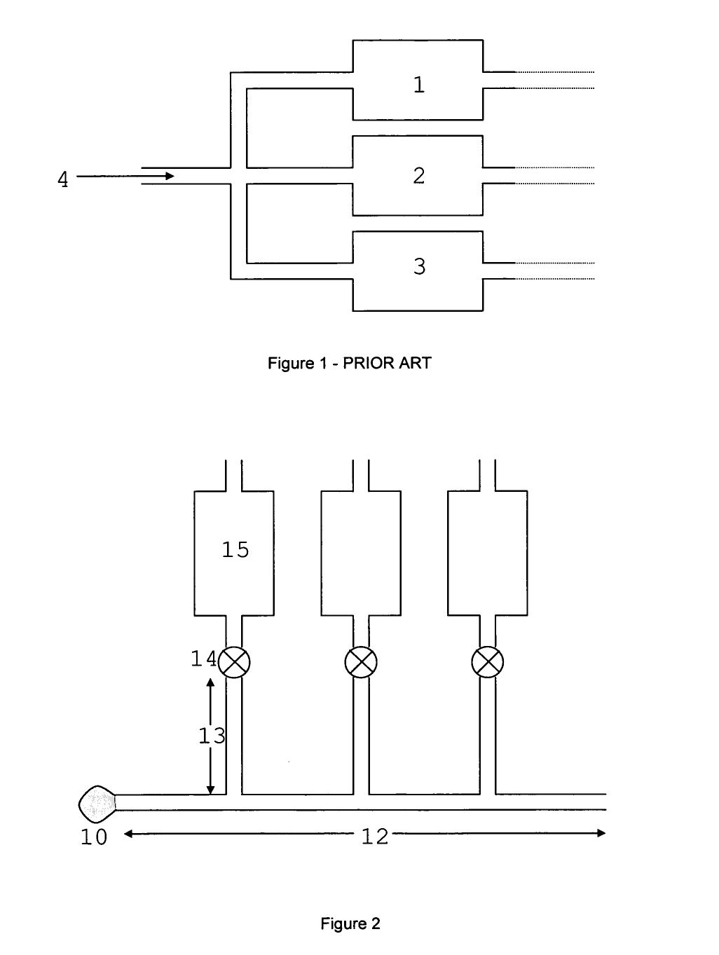

### DETAILED DESCRIPTION OF THE INVENTION

The inventors have recognised the difficulty in loading an analyte into a microfabricated system. This difficulty is compounded when loading an analyte through a common inlet port into reaction units arranged in parallel. In particular, the inventors have found it difficult to deliver a pre-determined volume into each reaction unit in a reliable, repeatable manner.

With some nucleic acid amplification techniques, internal standards are provided that allow quantitative analysis to be carried out on an analyte without knowing the analyte's volume or the ratio of the amounts of reagents (such as amplification enzymes) to the analyte. For example, in quantitative NASBA, one or more internal RNA standards may be used. If more than one standard is used, each standard may be chosen so that it can be easily detected at a particular concentration, for example at high, medium or low concentrations.

For multiple nucleic acid amplification reactions carried on a single analyte in a microfabricated system, different primers may be provided to different reaction units arranged in parallel in order to separately amplify and detect different nucleic acid sequences in the analyte. By using an internal standard, quantitative measurements can be made even when different, unknown analyte volumes are provided to the different reaction units.

However, the inventors have found that, for some applications, it is advantageous to be able to reliably deliver a pre-determined volume of an analyte to multiple sets of reaction units arranged in parallel.

For example, the inventors have recognised that sometimes an internal standard is not added before nucleic acid amplification is carried out. This could be for a number of reasons, such as there being no appropriate standard available for the relevant amplification technique, a concern that the standard could interfere with the amplification of the target nucleic acid, or a overly complex relationship between the detection of the standard and the detection of the target nucleic acid. To take another example, when a nucleic acid analyte is first extracted and purified from a clinical sample using a microfabricated system such as that described in WO 2005/073691 or WO 2008/149111 and delivered directly to the amplification units, it can be impractical to add another step to the purification sequence mixing internal standards with the analyte before loading it into the amplification units.

In addition, the inventors have recognised that even when an internal standard is added to an analyte before amplification, it can be advantageous for a pre-determined volume of analyte to be reliably and reproducibly provided to each reaction unit arranged in parallel connected to a common inlet port even though quantitative analysis is possible without knowing the exact volume added. For example, the actual concentration of a target molecule in a sample can sometimes be desired, for example, in the determination of the concentration of a nucleic acid, for example deriving from a virus, in drinking water. If the enzymes and/or primers used in the nucleic acid amplification are provided to the reaction units separately from the analyte, it can be necessary to know the actual volume of the analyte that is delivered to a reaction unit in order to carry out quantitative analysis to determine the concentration of the analyte in the original sample.

To take another example, it can be advantageous from a reproducibility perspective that the concentration of enzymes and primers to be the same in different analysis reactions carried out in parallel. Thus, when primers and/or enzymes are provided to a reaction unit independently from an analyte, for example if the primers and/or enzymes are provided in dry form in the reaction units, one way of ensuring that the primers and/or enzymes are provided in use during the amplification reactions in known concentrations is to provide the analyte in each reaction unit in a predetermined amount.

To take a further example, when carrying out a plurality of amplification reactions in a plurality of reaction units arranged in parallel, it can be advantageous from a practical viewpoint if the results of the different amplification reactions become available at the same time. Thus, the results of the nucleic acid amplification analysis can be obtained all at once rather than one at a time. This is beneficial in, for example, point-of-care diagnostic instruments comprising microfabricated devices for nucleic acid amplification so that the results of different diagnostic analyses become available at the same time.

Having made this recognition that it is sometimes desirable to reliably deliver a pre-determined volume of an analyte to multiple sets of reaction units arranged in parallel for whatever reason, the inventors have sought a system that reliably delivers a pre-determined volume of an analyte to multiple sets of reaction units arranged in parallel.

In particular, the inventors have recognised the versatility of the approaches described in WO 02/22265 and WO 03/060157 for carrying out nucleic acid amplification. However, the inventors have recognised that these systems suffer from a drawback that, if the loading of a sample into their systems is not carefully controlled, different amounts of sample can be loaded into the different sets of parallel reaction chambers. As a result, the inventors have sought a method of sample loading into a parallel set of reaction chambers connected to a common inlet that results in a more uniform volume of sample being loaded into each reaction chamber.

Accordingly, the present invention provides a method of metering an analyte in a microfabricated device into a plurality of reaction units arranged in parallel. The method comprises providing an integrated microfabricated device comprising a common inlet port, a supply channel connected to the common inlet port, and a plurality of reaction units connected in parallel to the common inlet port through the supply channel. Each reaction unit comprises a metering channel having a first end connected (preferably directly connected) to the supply channel and a second end, a first reaction chamber and a first valve connected (preferably directly connected) to the second end of the metering channel and separating the metering channel from the first reaction chamber. An analyte is then loaded into the common inlet port, and allowed to enter the supply channel, which may be directly connected to the common inlet port or may be separated from the common inlet port by a separate channel. From the supply channel, the analyte is allowed to flow into each of the metering channels up to each of the first valves. Then, any analyte remaining in the supply channel is drawn down the supply channel past and away from the first ends of each of the metering channels so that the aliquots of analyte loaded into the metering channels are isolated from one another. In this way, aliquots of analyte of pre-determined volume are provided in each of the metering channels. Finally, the aliquots of analyte metered in each metering channel are caused or allowed to pass through each valve into each of the first reaction chambers.

Accordingly, the first valves positioned at the end of the metering channels allows the metering of a pre-determined volume of an analyte into the first reaction chambers. In addition, the removal of any analyte remaining in the supply channel past and away from the ends of the first ends of each of the metering channels means that, when the analyte passes through the first valves, unknown amounts of additional analyte is not drawn up the metering channels from the supply channels.

As used herein, the term “connected” when applied to two parts of the device reflects that the two parts are in fluid communication with one another, for example by being directly joined to one another (i.e. are in direct physical contact). Parts that are “connected” to one another may be directly connected to one another. The term “directly connected” when applied to two parts of the device reflects that the two parts are directly joined to one another (i.e. are in direct physical contact). The term “downstream” means that, in use, a sample passes sequentially through the different parts of the device. While the term “downstream” includes within its scope two parts of the device being in direct fluid communication, it also includes within its scope when the two parts are separated by, for example, a valve or another part of the device.

As used herein, the term “valve” refers to a means for adjusting the flow of an analyte from one part of the apparatus to another part of the apparatus. In particular, the valve has the ability to allow flow, to prevent flow, and to adjust flow between these two extremes. Thus, a valve may completely prevent the flow of analyte from one part of the apparatus to another of the apparatus. Under an external stimulus, the flow of analyte through the valve can be affected. Many different types of valves are known for use in microfluidic applications, for example pneumatic valves, thermo-pneumatic valves, thermo-mechanical valves, piezoelectric valves, electrostatic valves, electromagnetic valves, electrochemical valves and capillary valves (also known as capillary force valves).

The term “caused” means that a change in the configuration of the system causes an event. For example, an external stimulus may be applied. Thus, preferably an analyte is caused to pass into the first reaction chambers from each metering channel, whereby an external stimulus is provided in the form of, for example, pressure from a pump connected to the outlet of the reaction units. The term “allowed” means that no change in the configuration of the system is required to cause an event. For example, if an analyte is allowed to enter the supply channel and then the metering channels, the analyte may enter the supply channel and metering channels by substantially only capillary forces resulting from, for example, the interaction of the analyte with the supply channel and metering channel.

The present invention also provides a device specifically adapted for use in this method. The device is illustrated in FIG. 2. A common inlet port (10) allows loading of an analyte into the supply channel (12), to which the common inlet port is connected. A series of reaction units are then connected to the supply channel. Although FIG. 2 shows three reaction units, the present invention is not limited to providing three reaction units. Instead, the present invention relates to the presence of a plurality of reaction units, i.e. two or more reaction units.

Each reaction unit comprises a metering channel (13) connected (preferably directly connected) to the supply channel. In FIG. 2, each metering channel is shown as branching/extending from the supply channel. The metering channel is directly connected to a first valve (14), which is itself connected to a first reaction chamber (15).

As understood by the person skilled in the art, the term “microfabricated” refers to devices that operate using the principles of micro-fluidics.

Methods of manufacturing microfabricated devices are also well-known to the person skilled in the art. For example, a microfabricated (microfluidic) device may be manufactured by hot embossing or injection moulding using a polymer. Alternatively, a microfabricated device may be manufactured using processes that are typically, but not exclusively, used for batch production of semiconductor microelectronic devices, and in recent years, for the production of semiconductor micromechanical devices. These processes can also be used for the manufacture of a die for use in a method of producing microfabricated devices using hot embossing or injection moulding.

For example, a microfluidic device or a die for the hot embossing manufacture of microfluidic devices may be manufactured by, for example, epitaxial growth (e.g. vapour phase, liquid phase, molecular beam, metal organic chemical vapour deposition), lithography (e.g. photo-, electron beam-, x-ray, ion beam-), etching (e.g. chemical, gas phase, plasma), electrodeposition, sputtering, diffusion doping and ion implantation. Typical crystalline semiconductor substrates may be used such as silicon or gallium arsenide, in which electronic circuitry may be integrated into the system by the use of conventional integrated circuit fabrication techniques. Combinations of a microfabricated component with one or more other elements such as a glass plate or a complementary microfabricated element may also be used.

For the mass production of microfabricated chips, injection moulding has the potential to be the most cost effective production technique. However, hot embossing is also an attractive approach to producing a microfabricated device because of its versatility: it does not require the use of an expensive injection moulding tool, and is adaptable so allows the production of small scale batches of chips.

Microfluidic devices are useful for carrying out analysis on samples containing only a small quantity of analyte. However, it becomes difficult to precisely control the dimensions of a microfluidic device if the dimensions are very small. Therefore, each metering channel in the microfabricated device preferably holds, in use, a volume of analyte of 100 μl or less, for example 10 μl or less, such as 5 μl or less. For example, each metering channel may hold, in use, 1 μl or less of analyte. For ease of manufacture, a minimum volume of each metering channel may be chosen as 1 nl, for example 10 nl, such as 100 nl. Thus, in one embodiment, each metering channel may hold between 10 nl and 10 μl of analyte, for example about 800 nl. Each of these values generally correspond to the volume of each metering channel from their connection point with the sampling channel (i.e. the first end of the metering channel) to the first valve (i.e. the second end of the metering channel).

The inventors have found that it is advantageous to allow each metering channel to be filled with the analyte by capillary forces. Without wishing to be bound by theory, it is thought capillary forces allow the metering channels to be filled in a more uniform manner than by using an external force such as that provided by a pump or a variable volume chamber. In particular, because the force driving the filling of the metering channel is an ‘internal’ capillary force rather than an external driving force, it is thought that the meniscus of the analyte rising up the metering channels is less likely to break, thereby having less of a chance of introducing air bubbles into the analyte loaded into the metering channels.

Accordingly, each metering channel is preferably filled by substantially only the action of capillary forces. Thus, no external driving force (e.g. a pump) need provide the driving force for the analyte to pass into the metering channels and up to the first valves. The term “substantially” reflects that there may be small inherent external driving force provided by, for example, weight of the analyte in an inlet port. While a small pressure may be provided by a pump (connected to the common inlet port or the outlet(s) of the reaction units) if it does not affect the smooth flow of the analyte filling the metering channels, the capillary forces and optionally any driving force provided by the weight of fluid may be sufficient on their own for fluid (e.g. water) to advance up and fill each metering channel.

In order to facilitate the filling of the supply channel and metering channels by capillary forces, preferably the metering channel is substantially uniform in cross-section. As such, preferably the ratio of the maximum area of cross-section to minimum area of cross-section of the metering channel, wherein the cross-section is measured in the direction perpendicular to the flow path of the analyte in use, is about 0.8 to 1, for example about 0.9 or greater, such as about 0.95 or greater, for example about 0.98 or greater. The inventors have found that, by providing a metering channel of substantially uniform cross-section, the filling of the metering channel by capillary forces may become more uniform, reproducible and controlled. In particular, if there is a large difference between the maximum and minimum areas of cross-section of the metering channel, the capillary force experienced by the analyte varies from the position of the analyte in the metering channel. This varying capillary force can result in unpredictable filling of the metering channels.

Furthermore, if there is a sudden change in the cross-section of the metering channel, the inventors have found that the analyte, when rising up the metering channel, may become ‘stuck’ part way up the metering channel and not rise up the valve at the end of the metering channel. Without wishing to be bound by theory, the inventors refer to the Concus-Finn condition. In particular, when an analyte meets a change a dimension of its flow path, filling past this change of cross-section is thought to occur when:

θ<π/2−α

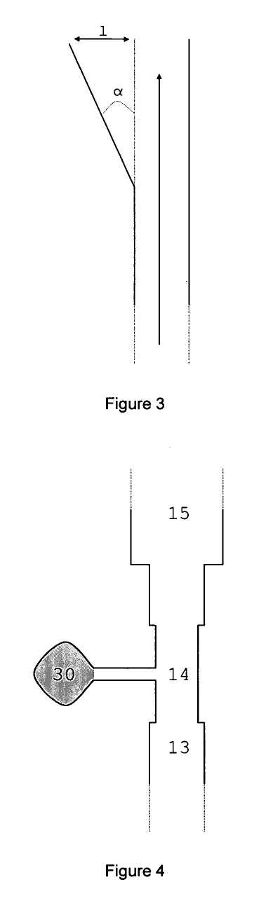

In this equation, θ is the static contact angle of the liquid on the surface of the device and α is the angle by which a dimension of the flow path measured perpendicular to the flow path (perpendicular to the axis of the metering channel) changes. The dimension may, for example, be the position of one of the sides, the base or the top of the channel that may make up the metering channel. The measurement of α is illustrated in FIG. 3, in which the change of dimension in one side of the flow path is illustrated as ‘I’. The flow path is shown by the arrow, which is parallel to the axis of the metering channel. In this Figure, it is shown how the change in a dimension of the metering channel is measured perpendicular to the flow path.

Accordingly, preferably at least one of the dimensions of the metering channel perpendicular to the flow path (e.g. those defining the width and depth of the metering channel) satisfies the condition θ<π/2−α at any point along the length of the metering channel. Preferably, the dimensions of the metering channel satisfies the condition θ<π/2−α_{AVE} along the length of the metering channel, where α_{AVE} is the average (mean) value of α at any point along the metering channel around the circumference of the metering channel. In particular, α_{AVE} may be calculated by measuring α at a number of evenly spaced points (e.g. at least 4 evenly spaced points, which would represent one for each side of the metering channel if the metering channel contains a top, bottom and two sides, for example 20 evenly spaced points) around the circumference of the metering channel. The number of evenly spaced points is chosen so that it gives a representative average of the value of α around the circumference of the metering channel. Preferably, all of the dimension of the metering channel perpendicular to the flow path satisfy the condition θ<π/2−α. Preferably, any of these conditions are satisfied along the whole length of the metering channel, from its connection with the supply channel up to the first valve.

θ, the static contact angle of the analyte on the surface of the device, can be measured by a static sessile drop method. It may be measured by placing a drop of the analyte onto a planar surface that replicates the surfaces of the device (i.e. it is made from the same material and has been treated in the same way). For example, contact angle goniometer may be used to take the measurement. The measurement may be taken at 25° C. and at 1 atmosphere pressure. For convenience, water may be used to determine a value of θ for a particular surface. For example, ultra-pure water may be used.

The surface of each metering channel may be hydrophilic, preferably along its entire length from its connection with the supply channel to the first valve. The supply channel may also be hydrophilic, preferably along its entire length.

The term “hydrophilic” and, as used later, the term “hydrophobic” take their ordinary meaning the art. Thus, a hydrophilic surface may have a static contact angle of water on its surface of less than 90° Preferably, the contact angle of the hydrophilic materials described herein is 0° (with water wetting its surface) to 60°, such as 5° to 45° or less, for example 35° or less, such as 30° or less, for example 25° or less, such as 20° or less, for example 15° or less. Conversely, a hydrophobic surface may have a static contact angle of water on its surface of 90° to 180°. Preferably, the contact angles of hydrophobic materials described herein is 110° to 170°, for example 125° or greater, such as 135° or greater. Preferably, the hydrophobic materials described herein are super-hydrophobic material. Accordingly, preferably the hydrophobic materials have a contact angle of water of 150° or greater, for example 155° or greater. In particular, with a contact angle further removed from 90°, the hydrophobic or hydrophilic nature of the material in question becomes greater and the desired effect from using a hydrophobic or hydrophilic substrate may increase.

In order to render the metering channels hydrophilic, a hydrophilic substrate may be provided. For example, the hydrated surface of a silicon substrate is hydrophilic. Another method of rendering the metering channels hydrophilic is to coat a non-hydrophilic substrate, for example a substrate made from a non-hydrophilic polymer, with a hydrophilic coating. Such coatings include polyethylene glycol (PEG), Bovine Serum Albumin (BSA), tweens and dextrans. The coating may have a typical thickness of up to 1 μm, preferably less than 0.5 μm.

Preferred dextrans are those having a molecular weight of 9,000 to 200,000, especially preferably 20,000 to 100,000, particularly 25,000 to 75,000, for example 35,000 to 65,000.

Tweens (or polyoxyethylene sorbitans) may be any of these available, for example, from the Sigma Aldrich Company.

PEGs are preferred as the coating means, either singly or in combination with other PEGs or other coatings. By PEG is embraced pure polyethylene glycol, i. e. of the formula HO-(CH₂CH₂O)ₙ-H, where n is an integer to afford a PEG having, for example, a molecular weight of from 200-10,000, especially 1,000 to 5,000; or chemically modified PEG in which one or more ethylene glycol oligomers are connected by way of homobifunctional group(s), such as, for example, phosphate linkers or aromatic spacers.

Particularly preferred is a polyethylene glycol known as P2263 (Sigma Aldrich Company) in which a polyethylene glycol chain is connected to another through aromatic spacers.

Preferably, in order to facilitate the loading of the channels by capillary forces, each metering channel may have a maximum area of cross-section of 20 mm², for example 10 mm², such as 5 mm², for example 2 mm², such as 1 mm². The cross-section is measured in the direction perpendicular to the flow path of the analyte in use, in other words perpendicular to the axis of each metering channel. The supply channel may independently have these preferred maximum areas of cross-section. However, for ease of manufacture, preferably each metering channel has a minimum cross-section of 0.01 mm², such as 0.1 mm². Thus, one preferred range of cross-section area is 0.01 to 5 mm².

Preferably, in order to facilitate the loading of the channels by capillary forces, the minimum ratio of the circumference of the metering channel to the area of cross-section of the metering channel is 6/d, wherein d is the maximum diameter of the metering channel. (It is noted that a channel a circular cross-section with a diameter of d has a circumference of πd and an area of cross-section of (d/2)²; therefore, the ratio of the circumference to area of cross section is only 4/d. Equally, the maximum diameter of a channel with a square cross-section is from one corner of the square to the opposite corner of the square. If this length is called d, then each of the sides of the square has a length of (√2)/2)d. Therefore, the diameter of the channel is 2(√2)d and the area of the cross-section of the channel is d²/2; the ratio of the circumference to area of cross section of the square is, as a result, 4(√2)/d=5.7/d). More preferably, the minimum ratio of the circumference of the metering channel to the area of cross-section of the metering channel is 8/d, such as 10/d. However, so that the metering channels are not distorted in one particular dimension in their cross-section, which can complicate fabrication and sample handling, preferably the minimum ratio of the circumference of the metering channel to the area of cross-section of the metering channel is 100/d, such as 40/d, for example 20/d. Thus, one preferred range of ratios is 6/d to 20/d.

In order to facilitate the filling of the supply channel and metering channels by capillary forces, each of the first valves connected to the end of each metering channel separating the metering channels from each first reaction chamber are preferably capillary valves.

The term “capillary valve” is well known to the person skilled in the art. It refers to a valve whose effect in restricting and/or allowing the flow of an analyte depends on the capillary pressure of the analyte.

There are several types of capillary valves. Electro-capillary valves take advantage of the change in surface tension of the surface of an analyte on applying an electric potential across the surface of the analyte. Thus, these capillary valves can be turned ‘on’ and ‘off’ by modulating an applied electric field across a channel. Thermo-capillary valves take advantage of the change in surface tension of the surface of an analyte on heating an analyte. Therefore, these capillary valves can be turned ‘on’ and ‘off’ by varying temperature.

The type of capillary valve that is especially preferred for use in the present invention is a passive capillary valve. These valves do not take advantage of some inherent variation in surface tension of an analyte; instead, an external force is applied to the analyte to force it through the valve. This external force may be applied by, for example, a pump attached to either side of the pump. Preferably, each valve is a passive capillary burst valve. In these valves, the valve retains an analyte until the pressure applied to the valve exceeds a particular pressure.

The passive capillary valves may comprise a hydrophobic constriction or a constricted section in a channel. The constriction may be in two dimensions, for example a narrowing in the width of a channel. Preferably, the constriction may be in three dimensions, for example a narrowing in the width of a channel and the shallowing of a channel. In particular, a constriction in three dimensions provides a greater constriction (and therefore a greater impedance) than a constriction in two dimensions.

For example, the constricted section may have a cross-section that is 80% or less of the area of the cross-section of the channel before the valve (e.g. of the metering channel, such as the maximum cross-section of the metering channel). The cross-section is measured in a direction perpendicular to the flow of the analyte in use. For example, the constriction may have a cross-section that is 70% or less in area, such as 60% or less in area, for example 50% or less in area. However, in order to control the force required to cause fluid to flow through the constriction, preferably the constricted section has a cross-section that is 1% or more of the cross-section of the channel before the valve, more preferably 5% or more, such as 10% or more. Thus, one preferred range of cross-sections is 5 to 80% of the cross-section of the channel before the valve, such as 5 to 80% of the maximum cross-section of the metering channel.

In some embodiments, the passive capillary valve may be manufactured from a separate material from the rest of the microfabricated device. Alternatively or additionally, the passive capillary valve may be rendered hydrophobic by coating it with a hydrophobic coating. Methods of rendering a surface hydrophobic are well known in the art. They include the physical deposition of a material onto a surface and the chemical deposition of a material onto a surface. For example, the hydrophobic material may be a fluoro-polymer, such as a polymer having an alkane backbone and having fluorine appending the backbone, or a polymer having one or more fluoro-alkyl monomer units, such as polytetrafluoro-ethylene (PTFE). A commercial example of a suitable fluoro-polymer is Teflon®. Alternatively or additionally, self-assembly of a surface active compound on a surface can render a surface hydrophobic. For example, silicon-containing compounds (for example, silicon halides, such as silicon chlorides and/or silicon alkyxoy compounds, such as silicon methoxy and/or ethoxy compounds) may react with a surface having nucleophilic sites to deposit a hydrophobic surface. The surface active compound may comprise an alkyl chain and/or a fluoroalkyl chain in order to enhance the hydrophobicity of the surface.

The burst pressure of a passive capillary valve formed from a constriction in a channel is determined by several factors. For example, the burst pressure depends on the extent of constriction of the channel and the hydrophobic nature of the constriction. The inventors have also found the burst pressure to depend on the actual fabrication of each valve. In particular, the inventors have found that, when the design of the valve at its outlet can influence both the burst pressure in terms of its size and in terms of its predictability.

If a burst capillary valve is provided as the first valve, its burst pressure is preferably 1 mPa or greater. In other words, when a pressure of 1 mPa or greater is applied to the valve, the analyte passes through the valve. Such a burst pressure conveniently allows the valve to perform its function in constricting or preventing the flow of liquid while allowing liquid to flow under suitable applied pressure. For convenience, the burst pressure may be measured at 25° C. with water, for example ultra-pure water. More preferably, the burst pressure is 5 mPa or more, such as 10 mPa or more. However, so that an excessive burst pressure is not required, preferably the burst pressure is 100 mPa or less, more preferably 50 mPa or less, such as 25 mPa or less. Thus, one preferred range of burst pressures is 1 to 100 mPa.

In order to spot a hydrophobic material onto a valve structure (e.g. a constriction in a channel) that has been microfabricated into a device, a structure such as that shown in FIG. 4 may be used. In FIG. 4, a reservoir (30) is provided adjoining the capillary valve and separate from the metering channel (13) and the first reaction chamber (15). When rendering a the constriction in a capillary valve hydrophobic, hydrophobic material in, for example, a solvent or in molten form, is placed in the reservoir (30) and allowed to flow and coat the surfaces of the capillary valve. Alternatively, a hydrophobic material may be spotted directly onto the valve structure without use of a dedicated reservoir adjoining the capillary valve.

Whatever method is used to provide a hydrophobic surface to the valves, preferably the hydrophobic surface extends into the first reaction chamber beyond the valve itself, for example beyond the constriction of the valve. The inventors have found that, by extending the hydrophobic area beyond the constricted section of the valve, the burst pressure of the valve can be more readily controlled and reproduced. Without wishing to be bound by theory, the inventors suspect that this is because, a sudden change from a hydrophobic surface to a hydrophilic surface at the valve outlet is avoided, thereby making the valve operation more predictable and reproducible.

The radius of curvature at the outlet of the valve in the section joining the constriction of the valve to the first reaction chamber is preferably 1 μm or more. In other words, preferably a sharp 90° corner (which is shown in FIG. 4) is not provided at the valve outlet. Instead, a smooth, curved surface transitioning from the valve structure to the first reaction chamber is provided. This smooth outlet may have a radius of curvature of preferably 100 μm or less. For example, the radius of curvature may be 5 μm or greater, such as 10 μm or greater, such as 20 μm or greater. The radius of curvature may be 50 μm or less, such as 30 μm or less, for example 20 μm or less. In one example, the radius of curvature at the outlet is 5 μm to 50 μm. In another example, the radius of curvature at the outlet is 5 μm to 20 μm. The inventors have found that, by providing a minimum radius of curvature, the burst characteristics of the valve become more controllable and predictable. In particular, the inventors suspect that, if the radius of curvature is too large, the energy required to wet the surface outside the valve becomes significant, especially when the hydrophobic surface extends beyond the outlet of the valve. However, a maximum radius of curvature is preferable in order to increase the burst pressure of the valves.

During the fabrication of the die for the device, the radius of curvature of the outlet of the device may be controlled by, for example, carrying out micromechanical machining of the valve outlets. One suitable technique is electro discharge machining (EDM) of the valve outlets.

The inventors have found the use of capillary valves in the present invention (especially passive capillary valves) to be versatile. In particular, the inventors have found that their use encourages smooth and even filling of the metering channels. This is thought to contribute in a more predictable volume of analyte being loaded into each metering channel. In addition, their use allows a pump to be connected to the outlet of each reaction unit to control the flow of the analyte through the capillary valves.

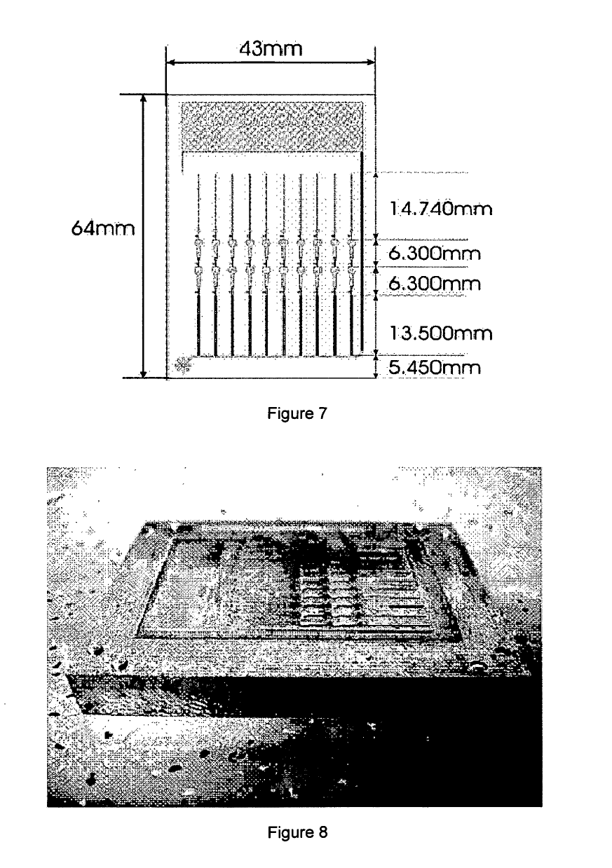

In order to facilitate the filling of the supply channel and metering channels by capillary forces, the common inlet port may preferably have a volume sufficient so that an analyte can be loaded into it and then allowed to be drawn by capillary forces down the supply channel. As an illustrative embodiment, the inventors of the present invention have found that designing a deep ‘star-shaped’ inlet port particularly facilitates capillary forces to operate. This configuration is apparent in FIG. 7. Alternatively or additionally, a sample loading chamber (21) may be provided downstream of the common inlet port but upstream of the supply channel. In such an arrangement, an analyte may be quickly loaded by, for example, injection and then allowed to pass out of sample loading chamber into the supply channel by, for example, capillary forces. If provided, the sample loading chamber may be provided with an optional valve (24, e.g. a capillary valve) separating it from the supply channel. Thus, reagents may be quickly loaded onto the chip and then allowed to flow into supply channel in a controlled manner by opening the valve 24.

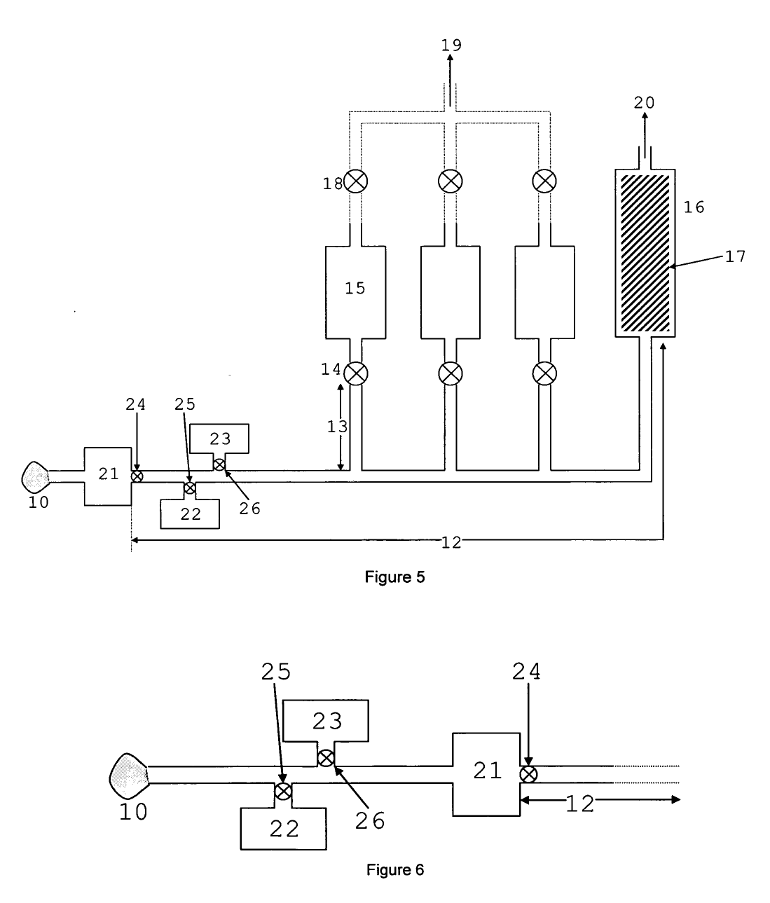

The supply channel may also be provided with one or more reagent storage chambers (22 and 23). For example, FIG. 5 shows the presence of two storage chambers. These storage chambers may be used to store reagents and may be provided with the reagents pre-loaded in the chambers. For example, one or more storage chambers may be used to store enzymes for nucleic acid amplification, solvents for increasing the specificity of a nucleic acid amplification reaction (e.g. a DMSO and/or sorbitol) by, for example, promoting the solvation of primers for nucleic acid amplification, and/or calibrators (i.e. internal standards) for calibrating nucleic acid amplification.

The storage chambers (22 and 23) may be separated from the supply channel by one or more optional valves (25 and 26). For example, one valve may be provided for each storage chamber. In use, the valves may be opened to allow reagents stored in the chambers to flow into the sample and mix with the sample as it is loaded into the device.

The storage chambers (22 and 23) may be microfabricated chambers contained on the same substrate as the rest of the device. Alternatively, the storage chambers (22 and 23) may be provided as pouches attached to connections connecting the pouches with the microfabricated system. A pouch is understood to be a flexible container with, preferably, a single opening that may act as an outlet. In use, the pouch decreases in volume according to the amount of reagent that has flowed out of the pouch. The emptying of reagent from the pouch may be facilitated by providing an external force to the pouch compressing the pouch, thereby increasing the internal pressure of the pouch compared to the pressure of the system into which the contents of the pouch is being dispensed.

The device may also be provided with simply valves (25 and 26) without storage chambers (22 and 23) but configured to be connected to a storage chamber such as a pouch. In use, containers of reagents (e.g. pouches) may be connected to the valves and loaded into the device by opening of the valves. This has the advantage that, for example, the device may be pre-fabricated but the reagents may be provided fresh at or close to the point of use. This is advantageous especially for liquid reagents, for example enzymes for nucleic acid amplification in solution, that may have a limited shelf-life.

It is noted that, if the system is also provided with a sample loading chamber, one or more of these storage chambers may be located downstream of the sample loading chamber (as shown in FIG. 5) and/or upstream of the sample loading chamber (i.e. in between the sample inlet (10) and the sample loading chamber (21).

In particular, a mixing unit (21) may be provided downstream of the reagent storage chambers (23 and 24). Thus, a sample may be loaded into the sample inlet (10), pass down a channel into which reagents are released from one or more reagent storage units (22 and 23) optionally through valves (25 and 26), and then pass to a mixing unit, for example a chamber, in which the reagents fully mix with the sample. The mixing unit may also take on the role as a sample loading chamber as described previously.

The presence of the mixing unit may be advantageous in order to obtain a uniform composition. Thus, the same composition is loaded into each of the reaction units, increasing the reliability and reproducibility of the device.

Preferably, the device does not contain means for processing or purifying the sample in between the sample inlet and the metering channels. In other words, the composition of the sample flowing into and up the metering channels is preferably the same as the composition of the sample entering the sample inlet (20), just having been optionally mixed with one or more reagents. This simplifies the design of the device and allows processing of a sample to obtain an analyte suitable for, for example, nucleic acid amplification, to be undertaken in a dedicated system or device.

The supply channel (12) may be provided with its own outlet, independent of the outlets of the reaction units. This facilitates, in use, an analyte being loaded into the common inlet port, allowed to pass down the supply channel and be drawn into the metering channels extending from the supply channel and then, once all of the metering channels have been filled, any remaining analyte to flow past and away from the ends of the metering channels extending from the supply channel. This outlet may connect with a waste unit, for example by being attached to it or feeding into it.

The device may comprise a waste unit. The waste unit is shown in FIG. 5 as (16). This waste unit may be a chamber connected to the outlet of the supply channel. The waste unit may preferably comprise a wicking material (17) that, in use, draws analyte out of and away from the outlet of the supply channel. In particular, the presence of a wicking material in the waste unit has been found to facilitate the drawing up of excess liquid through the supply channel, thereby facilitating the isolation of the plugs of liquid that are exposed at the end of the metering channels once the excess liquid has been drawn into the waste unit. The wicking material may, for example, be a filter material, for example cotton materials, such as cotton linter. Alternatively or additionally, the waste unit may be connected to a pump through an outlet (20) to aid the removal of the analyte from the supply channel once all of the metering channels have been filled.

Each of the reaction units may contain one or more reagents. The reagents may be pre-loaded into the reaction units during the manufacture of the device. The reagents may be selected to carry out any suitable biological or chemical reaction such as, for example, enzyme reactions, immuno reactions, sequencing, hybridisation. For example, the reagents may comprise amplification primers, enzymes and nucleotides. In one preferred embodiment, the reagents comprise at least primers for nucleic acid amplification, which may preferably be pre-loaded in the first reaction chamber. Alternatively, or additionally, the reagents may preferably comprise enzymes for nucleic acid amplification, which may preferably be pre-loaded into the second reaction chamber. The amplification primers and the nucleotides may, for example, be pre-loaded into the first reaction chamber. The reagents may also comprise means for detecting the amplification product, for example a molecular beacon probe oligonucleotide.

Preferably, primers and/or enzymes for nucleic acid amplification are provided in the reaction chambers of the device. Preferably, the primers and/or enzymes are for isothermal nucleic acid amplification, in which the sample is held at a constant temperature during amplification. In particular, the use of capillary forces to load chambers and control a sample on a chip may be much more controllable at a fixed temperature rather than at the fluctuating temperature used for thermal cycling. In one specific example, the reagents may comprise NASBA primers, ribonucleoside and deoxyribonucleoside triphosphates, enzymes for carrying out a NASBA reaction and molecular beacon probe oligonucleotide.

The reaction units may comprise a second reaction chamber. The second reaction chamber may be separated from the first reaction chamber by a valve. Preferably, this valve is of the same design as the first valve. Thus, the valve may be a passive capillary valve. A third valve may be connected to the outlet of the second reaction chamber. The second reaction chamber may be pre-loaded with amplification enzymes, for example enzymes for carrying out a NASBA reaction.

Preferably each reaction unit has its own outlet. Preferably, at least one valve separates the first reaction chamber from the outlet of the reaction unit. If the valve is a passive capillary valve, preferably the valve has a burst pressure that is at least twice the burst pressure of any other passive capillary valve, for example at least four times the burst pressure, such as at least five times the burst pressure.

Preferably, every outlet of the reaction units are connected to a single pump. The inventors have found that, in use, this can allow for the more controlled filling of the reaction chambers than compared to separate pumps controlling the individual reaction units. The single pump may be configured so that, in use, it is capable of actuating fluids in all of the reaction units through its connections to the outlets of the reaction units. It may be un-connected to any other part of the microfabricated device, or it may be connected to the waste unit to facilitate the drawing up of any excess fluid up the supply channel into the waste unit. It is also possible to provide a separate pump may to facilitate the drawing up of any excess fluid up the supply channel into the waste unit.

Preferably, the microfabricated device is integrated. In other words, the component parts of the device (e.g. the common inlet, supply channel, metering channels and reaction chambers) are formed on the same substrate. In some embodiments, the waste unit, if present, may also be formed on the same substrate as the other parts of the device, although in other embodiments it may be provided separately.

Preferably, means are provided for heating the contents of the first chamber to a constant temperature. Preferably, means are provided for heating the contents of the first chamber to a temperature of from 60 to 70° C., more preferably from 63 to 67° C., still more preferably about 65° C. Preferably, means are provided for heating the contents of the second chamber to a constant temperature. Preferably, means are provided for heating the contents of the second chamber to a temperature of up to 41.5° C., more preferably less than or equal to 41° C. In one embodiment, the means for heating the contents of the first chamber and the second chamber are the same means (e.g. the same heating element).

In order to monitor and/or maintain the desired temperature, a temperature controller may be provided associated with the first reaction chamber. The controller may comprise a first temperature sensor positioned adjacent to the first reaction chamber.

Preferably, the first temperature controller comprises a first controllable electric heat source (for example an electrical resistor element) positioned adjacent to the first reaction chamber and the second temperature controller comprises a second controllable electric heat source (for example an electrical resistor element) positioned adjacent to the second reaction chamber.

The system may thus preferably include integrated electrical heaters and temperature control.

Peltier element (s) and/or thermocouple (s) may be used to maintain the sample at the desired temperature in the reaction unit, preferably to within 0.5° C. In particular, thermocouples may be used to measure the temperature of the first and second chambers, wherein the thermocouples are linked by one or more feedback circuits to Peltier elements for heating the sample to the desired temperature in the first and second chambers.

A thermal barrier may advantageously be provided to substantially thermally isolate the different parts of the reaction unit from one another. For example, if a second reaction chamber is provided, a thermal barrier may be provided between the first and second reaction chambers. The thermal barrier may simply comprise a portion of a channel that spaces a first reaction chamber from a second reaction chamber. Different portions of the channel may define one or both of the reaction chambers.

The device may be provided with an optical interface for excitation and/or detection purposes.

Accordingly, if optical observations of the contents of the reaction unit are required, then at least one wall defining the relevant part of the reaction unit (e.g. the second reaction chamber) comprises an optically transparent substance or material, for example a polymeric material or glass. Preferably, the system comprises at least one optical source arranged for exciting fluorescence in material contained within the second reaction chamber, and at least one optical detector, arranged to detect said fluorescence. For example, molecular beacon probes may be provided in the second reaction chamber to detect one or more target nucleic acid sequences.

These probes fluoresce when in the presence of target nucleic acid sequences, thereby enabling detection and quantification of such sequences. The system thus provided amplification combined with optical- fluorescence detection. However, other detection methods could be used, for example, using impedance measurements. Preferably, the optical source is provided by one or more light emitting diodes, and the optical detector comprises at least one avalanche photodiode. However, other sources and detectors could also be used. For example the optical detector could comprise at least one photomultiplier tube.

Preferably, a bandpass filter is provided to filter the light impinging on the detector, in particular to filter out light emitted by the optical source. A micro-lens may be provided to direct the fluorescence onto the detector.

As will be appreciated, although the invention has been exemplified above in relation to first and second reaction chambers and in relation to certain temperatures of heating, the invention is not limited thereto. In particular, third, fourth, fifth or more reaction chambers may be provided in each reaction unit. The reaction units may also be configured to heat or cool their contents to any temperature required of the particular reaction protocol, for example for nucleic acid amplification.

The system or at least a master version thereof may be formed from or comprise a semiconductor material, although dielectric (eg glass, fused silica, quartz, polymeric materials and ceramic materials) and/or metallic materials may also be used. However, preferably, the system is formed from a plastic substrate. This may be formed using a semiconductor (e.g. silicon) master.

Examples of semiconductor materials for use as substrates or as master materials include one or more of: Group IV elements (i. e. silicon and germanium); Group III-V compounds (eg gallium arsenide, gallium phosphide, gallium antimonide, indium phosphide, indium arsenide, aluminium arsenide and aluminium antimonide); Group II-VI compounds (eg cadmium sulphide, cadmium selenide, zinc sulphide, zinc selenide) ; and Group IV-VI compounds (eg lead sulphide, lead selenide, lead telluride, tin telluride). Silicon and gallium arsenide are preferred semiconductor materials. The system may be fabricated using conventional processes associated traditionally with batch production of semiconductor microelectronic devices, and in recent years, the production of semiconductor micromechanical devices.

Such microfabrication technologies include, for example, epitaxial growth (e.g. vapour phase, liquid phase, molecular beam, metal organic chemical vapour deposition), lithography (e.g. photo-, electron beam-, x-ray, ion beam-), etching (e.g. chemical, gas phase, plasma), electrodeposition, sputtering, diffusion doping, ion implantation and micromachining. Non-crystalline materials such as glass and polymeric materials may also be used. Where polymeric materials are used, fabrication may be effected using conventional processes for manipulating plastics/polymeric materials such as, for example, injection moulding. Examples of polymeric materials include PMMA (Polymethyl methylacrylate), COC (Cyclo olefin copolymer), polyethylene, polypropylene, PL (Polylactide), PBT (Polybutylene terephthalate) and PSU (Polysulfone), including blends of two or more thereof.

Combinations of a microfabricated component with one or more other elements such as a glass plate or a complementary microfabricated element may be used.

The device may be designed to be disposable after it has been used once or for a limited number of times. This is an important feature because it reduces the risk of contamination.

The device may be incorporated into an apparatus for the analysis of, for example, biological fluids, dairy products, environmental fluids and/or drinking water. Again, the apparatus may be designed to be disposable after it has been used once or for a limited number of times.

The microfabricated system/apparatus may be included in an assay kit for the analysis of, for example, biological fluids, dairy products, environmental fluids and/or drinking water, the kit further comprising means for contacting the sample with the device. Again, the assay kit may be designed to be disposable after it has been used once or for a limited number of times.

The microfabricated system as herein described may be a nanofabricated device.

Preferably, and in particular if optical observations of the contents of the second reaction chamber are required, the cover overlying the measurement part of the reaction unit is made of an optically transparent substance or material. For example, glass, Pyrex or transparent polymers may be used.

If the system includes an optical source arranged for exciting fluorescence in material contained within the second reaction chamber, and an optical detector, arranged to detect said fluorescence, the surface in the second reaction chamber is preferably optically smooth. It has been found that the surface roughness of the wall (s) defining the second chamber on which light may be incident should be less than approximately 1/10th of the wavelength of the light.

A typical operation of the device of the present invention according to the method of the present invention will now be described. In this description, preferred aspects are intended to applicable across the breadth of the invention without necessarily requiring any of the other features described unless otherwise stated.

In use, a sample comprising an analyte in loaded into the common inlet port. This may be carried out in any appropriate manner, for example by injection. Alternatively, or additionally, the common inlet port may be connected to a device for extracting and purifying an analyte from a sample taken from, for example, a patient. For example, such a device may be configured to extract and purify nucleic acids from cells. Suitable examples of such systems are described in WO 2005/073691 and WO 2008/14911.

The analyte may be a nucleic acid sample. This sample may be derived from, for example, a biological fluid, a dairy product, an environmental fluids and/or drinking water. Examples include blood, serum, saliva, urine, milk, drinking water, marine water and pond water. For many complicated biological samples such as, for example, blood and milk, it will be appreciated that before one can isolate and purify DNA and/or RNA from bacterial cells and virus particles in a sample, it is first necessary to separate the virus particles and bacterial cells from the other particles in sample. It will also be appreciated that it may be necessary to perform additional sample preparation steps in order to concentrate the bacterial cells and virus particles, i. e. to reduce the volume of starting material, before proceeding to break down the bacterial cell wall or virus protein coating and isolate nucleic acids. For example, when the starting material consists of a large volume such as an aqueous solution containing relatively few bacterial cells or virus particles, it may be advantageous to concentrate the sample. This type of starting material is commonly encountered in environmental testing applications such as the routine monitoring of bacterial contamination in drinking water.

After loading, the sample may pass down a channel to a sample loading chamber (21). This chamber may be of sufficient volume to hold all of the sample. Once loaded into sample loading chamber, a valve (24) at an outlet of the sample loading chamber at the connection between the sample loading chamber and the supply channel may be opened to allow flow of the sample to the supply channel.

Before (or after) reaching the sample loading chamber, reagents may be added to the sample from one or more reagent storage chambers (22 and 23). For example, the reagent storage chambers may be pouches that are attached to the device through valves (25 and 26). The use of pouches in this way allows reagents to be provided to the system fresh at the point of use. In use, the pouches may be compressed in order to force reagent stored in the pouches into the channel connecting the sample inlet with the sample loading chamber. The sample loading chamber may also be in the form of a mixing unit to fully mix the reagents from the reagent storage chamber(s) with the sample. The mixing unit may be provided with mixing means. Mixing means include an elongated channel, for example a sinuate channel that mixes by creating turbulent flow and/or a chamber filled with beads (for example, magnetic beads) that may be agitated (for example by a magnetic field) in use.

For example, reagents including enzymes for nucleic acid amplification may be added to a sample. Alternatively, or additionally, one or more internal standards having known calibration curve(s) with respect to the nucleic acid sequences being amplified may be added. Alternatively, or additionally, fluids for the promotion of selective amplification, for example DMSO and/or sorbitol, may be added to the sample.

It is noted that reagents storage chambers may also be located downstream of the sample loading chamber but upstream of the reaction units. It is also noted that the device may not comprise a sample loading chamber and/or reagent storage chambers.

It is also conceived that reagent storage chambers (22 and 23) may also be provided connected directly to the mixing unit/sample loading chamber.

Whether or not a sample loading chamber and/or reagent storage chambers are provided, the analyte is allowed to enter and flow up a supply channel. In one embodiment, no outside force is provided, so the analyte flows up the supply channel by means of capillary forces only and optionally under the weight of the sample. Alternatively, a pump can provide a force to cause the sample to flow up the supply channel.

While flowing up the supply channel, the analyte passes past the first ends of the metering channels. In doing so, the analyte is caused by substantially only capillary forces to flow into the metering channels. The metering channels are substantially uniform in cross-section in order to facilitate the loading by capillary forces.

Having entered the metering channels, the analyte flows up to the second ends of the channels. Here, the analyte encounters a valve (preferably a capillary valve), where the flow of the analyte is halted.

Once all the metering channels have been filled, any of the sample that remains in the supply channel is drawn up the supply channel to the waste unit (16). This may be caused by capillary forces. It may be also be facilitated by the presence of a wicking medium (17) in the waste unit and/or by a pump attached to the outlet (20) of the waste unit.

The metering channels now contain a pre-determined volume of sample. Preferably, each metering channel contains approximately the same amount of pre-determined volume of sample.

Next, if the valves are passive capillary valves, a pump connected to the outlets of the reaction units applies a pressure above the burst pressure of the first valve in order to cause the sample to pass through the first valves. The pump may apply the pressure in a burst to reduce the chance of the sample to pass immediately out of the first reaction chamber and through the second valve. Other means may be provided to cause the sample to pass into the first reaction chamber through the first valves if the valves are not passive capillary valves.

Finally, nucleic acid amplification is carried out on the sample now contained in pre-determined quantities in the reaction chambers. The amplification may be isothermal nucleic acid amplification. Amplification may be followed by detection of the amplified products in the microfabricated system.

In the above description, various aspects of the microfabricated device of the present invention have been described in relation to the method of using the device and various aspects of using the device have been described in relation to the device itself. It is intended that the preferred aspects of the device of the present invention are also preferred aspects of the method of the present invention and vice versa so that, when an aspect of the device has been described in relation to the method, it is intended that this aspect is also a preferred aspect of the device of the present invention itself, and vice versa.

### Examples

To illustrate the invention, the manufacture of a device according to the present invention comprising a plurality of reaction units, each reaction unit comprising a metering channel, a first valve, a first reaction chamber, a second valve, a second reaction chamber and a third valve connected to one another in that order will now be described. The use of this device in NASBA will then be described.

Hot embossing was used to manufacture a batch of devices. To simplify the production process, a semi-finished part possessing the outer geometries and an already grinded surface was used for the production of the die. To allow a good machinability, the material of this semi-finished part was chosen to be a special stainless steel (German nomenclature 1.2312).

The first step of the manufacturing of the die was the construction of a CAD 3D model with all relevant dimensions, as shown in FIG. 7. This model was transformed into a CNC program file, allowing the milling of the die. Different end mills were used to structure the die, going down to a diameter of 0.3 mm. The milling was followed by electro-discharge machining (EDM) to reduce the radius of curvature at the valve outlets. Within the EDM process, a voltage is applied to the work piece and an electrode, all kept inside a dielectric fluid. When the distance between work piece and electrode becomes to narrow, some small amount of material of both pieces is molten and blasted away.

First, two copper electrodes were made by wire EDM, possessing the negative form and hence no radii at the significant sites of the structures at the valves. With these electrodes, the surplus material of the actual die was removed by sink EDM.

After the EDM process the die was used to hot emboss the prototype NASBA chips. The die is shown in FIG. 8.

The die was fitted with a few positioning pins and a spacer of 2 mm height, leaving only the chip surface of 64×43 mm² open. After inserting a blank chip of 2 mm thick COG polymer into this gap, the hot embossing form was heated up. After closing the two halves (the other side of the hot embossing die is just plain), the tool was put under pressure and so the structure of the die was pressed into the soft polymer. After cooling the chip was taken out of the tool and the surplus material, collecting at the edges of the 64×43 mm² area, was removed by milling.

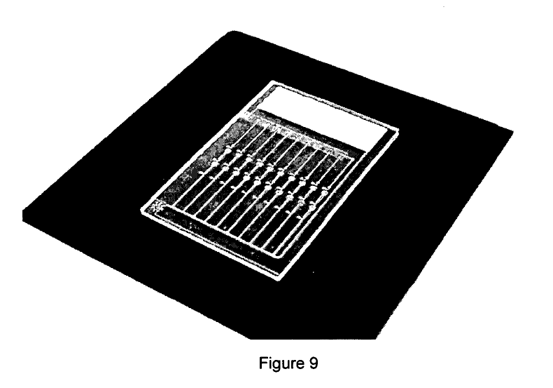

An image of a manufactured prototype chip is shown in FIG. 9. Absorbing filter paper resides inside the waste chamber. The chip is sealed with microencapsulated adhesive foil manufactured by 3M.

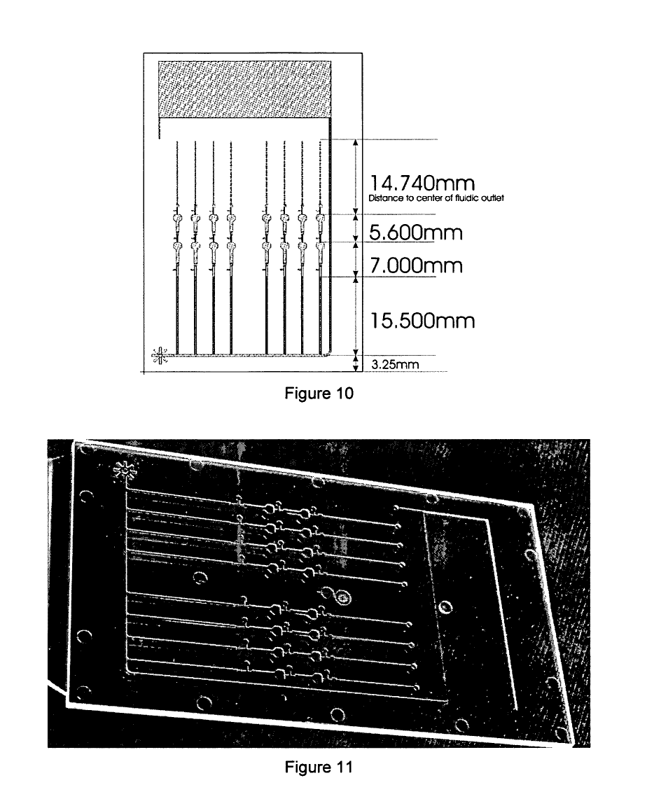

Injection moulding was used to manufacture a second batch of devices. The dimensions of these devices are shown in FIG. 10 and a representative chip manufactured by injection moulding is shown in FIG. 11.

The injection-moulded chips were then tested. In total, 65 chips were evaluated with respect to sample metering. Each chip had 8 channels, so a total of 520 channels were tested. 97.7% of the metering channels performed their metering function, with a high volumetric accuracy (better than 5%). The few observed faulty cases did not result from chip design but to the current manual handling procedures of the chip during its preparation.

NASBA was carried out in the reaction units. In particular, the units were used to detect human papillomavirus (HPV), the virus implicated in cervical cancer. Specifically, HPV strains 16, 31 and 33 were detected. In some cases, all of the NASBA reagents apart from the NASBA enzymes were premixed and spotted in the first reaction chambers. In other cases, the primers and probes were spotted and dried in the second reaction chamber. In either case, the NASBA enzymes were spotted in the second reaction chambers before use.

The reagents for NASBA included the primers for the specific strains, the individual nucleotide bases required for amplification and, because the amplified product was intended to be detected by fluorescence detection of a molecular beacon to the amplified product, appropriate molecular beacons.

In use, the separate aliquots of analyte were then passed into the second reaction chambers, in which they were heated to 41° C. Amplification product was detected by fluorescence of molecular beacons. Detection was carried out using a single excitation wavelength and by monitoring a single emission wavelength. As a control, macroscale experiments were also carried out on the same analyte samples.

A comparison of the results of the nucleic acid amplification in the microfabricated system showed that the microfabricated system could reliably replicate the results obtained when amplification was carried out on the macroscale.

1. A method of metering an analyte in a microfabricated device into a plurality of reaction units arranged in parallel and connected to a common inlet port, the method comprising:
providing a microfabricated device comprising:
(a) a common inlet port,
(b) a supply channel connected to the common inlet port, and
(c) a plurality of reaction units connected in parallel to the supply channel, each reaction unit comprising:
(c1) a metering channel having a first end connected to the supply channel and a second end,
(c2) a first reaction chamber, and
(c3) a first valve positioned at the second end of the metering channel and separating the metering channel from the first reaction chamber;
loading an analyte into the common inlet port,
allowing the analyte to enter the supply channel and then into each of the metering channels up to each of the first valves,
causing or allowing any analyte remaining in the supply channel to be drawn down the supply channel past and away from the first ends of each of the metering channels; and then
causing or allowing the analyte metered in each metering channel to pass through each valve into each of the first reaction chambers.

2. The method of claim 1, wherein the analyte is drawn into each metering channel by substantially only capillary forces and preferably is drawn into the supply channel by substantially only capillary forces.

3. The method of claim 1, wherein the supply channel has a first end connected to the common inlet port and a second end, and wherein the integrated microfabricated device further comprises a waste unit connected to the second end of the supply channel, and wherein any analyte remaining in the supply channel after the loading into the metering channels is allowed to flow through the supply channel into the waste unit.

4. The method of claim 3, wherein the waste unit contains a wicking medium, and wherein the volume of the analyte loaded into the common inlet port is sufficient to fill the metering channels and the supply channel up to the waste unit.

5. The method of claim 4, wherein the wicking medium draws the analyte remaining in the supply channel after the loading into the metering channels up the supply channel into the waste unit.

6. A microfabricated device for carrying out nucleic acid sequence amplification on an analyte, the device comprising:
(a) a common inlet port,
(b) a supply channel connected to the common inlet port, and
(c) a plurality of reaction units connected in parallel to the supply channel, each reaction unit comprising:
(c1) a metering channel having a first end connected to the supply channel and a second end,
(c2) a first reaction chamber, and
(c3) a first valve positioned at the second end of the metering channel and separating the metering channel from the first reaction chamber.

7. The device of claim 6, wherein the first valve is a capillary valve.

8. The device of claim 7, wherein each reaction unit further comprises a second capillary valve upstream of the first reaction chamber, wherein the burst pressure of the second capillary valve is equal to or greater than the burst pressure of the first capillary valve.

9. The device of claim 8, wherein each reaction unit further comprises an outlet, and the outlets of each reaction unit are connected to a single pump.

10. The device of claim 6, wherein at least the supply channel and each of the metering channels is treated to be hydrophilic.

11. The device of claim 6, wherein the surface of each of the metering channels has a contact angle of water of 50° or less.

12. The device of claim 6, wherein the supply channel has a first end connected to the common inlet port and a second end, and wherein the microfabricated device further comprises a waste unit connected to the second end of the supply channel.

13. The device of claim 12, wherein the waste unit contains a wicking medium.

14. The device of claim 6, wherein each reaction unit further comprises:
(c4) a second reaction chamber; and
(c5) a second valve separating the first reaction chamber from the second reaction chamber.

15. The device of claim 6, wherein reagents for nucleic acid amplification are provided in each of the reaction units.

16. The device of claim 6, wherein primers for nucleic acid amplification are provided in each first reaction chamber.
